(19) United States
(12) Patent Application Publication
(10) Pub. No.: US 2021/0150939 A1
(43) Pub. Date: May 20, 2021

(54) User-Friendly Ingredient Label

(71) Applicant: The Procter & Gamble Company, Cincinnati, OH (US)

(72) Inventors: Shawn David McCONAUGHY, Cincinnati, OH (US); Thomas Arthur STURGIS, Mason, OH (US); James Daniel GALLAGHER, Cincinnati, OH (US)

(21) Appl. No.: 16/952,402

(22) Filed: Nov. 19, 2020

Related U.S. Application Data
(60) Provisional application No. 62/937,460, filed on Nov. 19, 2019.

(51) Int. Cl. G09F 3/02

(52) U.S. Cl. CPC G09F 3/02; G09F 2003/0273

(57) ABSTRACT

A personal care product that includes a personal care composition disposed in a package and a user-friendly ingredient label disposed on the package. The user-friendly ingredient label includes a first portion containing a formal ingredient name for each ingredient in the product and a second portion containing at least one of an informal ingredient name and additional ingredient information related an ingredient in the product. The ingredient label configuration allows a product manufacturer to comply with governmental regulations and/or industry standards for labeling products and still provide additional salient ingredient information that is desired by consumers for the ingredients in the product.

### FIELD

The present disclosure relates, generally, to a user-friendly ingredient label for use on product packaging. More specifically, the present disclosure relates to an ingredient label that lists formal ingredients names for at least some of the product ingredients in a first portion of the label and a corresponding informal ingredient name and/or additional ingredient information in a second portion of the ingredient label.

### BACKGROUND

Certain consumer products such as cosmetics, food, and dietary supplements are required to list the ingredients of the product on the package. In many countries, ingredient labeling requirements for food, drugs, and cosmetic products may be subject to governmental regulation. For example, product labeling requirements may include size and placement of the label, information that must listed on the label, uniform ingredient names (e.g., INCI names), order of ingredients, and type size and prominence.

The use of standardized ingredient names on the label of cosmetic products such as facial moisturizers, shampoos, and other personal care products can help provide uniformity in product manufacturing and improve consumer safety. However, standardized ingredient names are not necessarily unique to a specific material and may not differentiate ingredients based on the source of the ingredient. For example, the INCI name “Butylene Glycol” does not indicate if the material is sourced from traditional petroleum-based feedstocks or newer, more consumer-preferred bio-based feedstocks. A given INCI name may encompass more than one exact chemical. For example, “Acrylates Copolymer” includes a wide range of different monomers copolymerized with acrylic acid and/or methacrylic acid. Providing additional information with regard to the monomer content of an “acrylate copolymer” may be desired by at least some consumers. Advancement in smart phone apps has enabled consumer to readily scan ingredient statements. However, the information provided to the consumer by these apps is still limited by the utility of current INCI names. In light of the limited information provided by formal ingredient names, consumers may not recognize what the ingredients in a product are and/or what they do.

To raise consumer awareness of a particular ingredient, some cosmetic manufacturers may provide additional ingredient information on a product website accessible via the Internet. For example, the website may provide the common name and/or properties of the key ingredients for a particular product (i.e., the ingredient(s) providing the claimed cosmetic benefit). In a specific example, the website may indicate that retinyl palmitate is “Vitamin A”. In another specific example, the website might provide an ingredient list for a product that includes “Nymphaea alba extract” and indicate in stylized or prominent lettering that “water lily extract is an excellent source of antioxidants and nutrients to replenish the skin”. While such websites may provide a suitable source of additional ingredient information for consumers who are more actively researching a product or specific ingredient or shopping online, they are of little use to a consumer in a brick-and-mortar retail environment (i.e., at a physical store shelf). Thus, it would be desirable to provide additional ingredient information desired by consumers on the product packaging such that it is accessible by the consumer in a brick-and-mortar retail environment.

Providing additional ingredient information on the ingredient label and/or packaging of a personal care product such as a facial moisturizer may be particularly problematic since it is not uncommon for such products to be sold in relatively small packages. For example, some facial moisturizer products may be sold in a small box (e.g., secondary packaging) that has less than 12 square inches of useable surface area. Due to the relatively small size of the box, there may not be sufficient space on the packaging to include additional ingredient information. And while it may possible to increase the package size, this is not always desirable, since some consumers prefer smaller packaging for product portability and reduced environmental impact.

To provide consumers with at-shelf ingredient information, some cosmetic product manufacturers may include additional ingredient information on the ingredient label, but only for the key ingredients in the product. For example, a product that has tocopherol acetate listed in the ingredients may be followed by “Vitamin E” in parentheses. In another example, the key ingredients in the product may be listed in a separate portion of ingredient label and/or product packaging, for example, under the heading “Active Ingredients” or “Key Ingredients”.

Accordingly, it would be desirable to provide additional ingredient information to consumers that includes a more recognizable name for the ingredient and/or additional ingredient information. It would also be desirable to provide the additional information in a way that is compatible with current packaging for consumer products, especially consumer products that are sold in relatively small packages, such as skin care products.

### SUMMARY

Disclosed herein is a personal care product comprising a personal care composition disposed in a package and an ingredient label disposed on the package. The ingredient label includes a first portion containing a formal ingredient name for each ingredient in the product and a second portion containing at least one of an informal ingredient name and additional ingredient information that corresponds to the formal ingredient names in the first portion. In some embodiments, the ingredient label may further comprise a third portion that lists formal ingredient names for ingredients that are present in the product at less than a threshold amount (e.g., 0.1%).

### BRIEF DESCRIPTION OF THE DRAWINGS

FIG. 1 shows a conventional ingredient label for a cosmetic skin care product.

FIG. 2 shows an example of an embodiment of the inventive ingredient label.

FIG. 3 shows an example of an embodiment of the inventive ingredient label.

FIG. 4 shows an example of an embodiment of the inventive ingredient label.

FIG. 5A shows a conventional ingredient label used in a consumer test.

FIG. 5B shows an example of an embodiment of the present ingredient label used in a consumer test.

FIG. 6A illustrates the results of the consumer test for the conventional ingredient label.

FIG. 6B illustrates the results of the consumer test for the inventive label.

### DETAILED DESCRIPTION

At least some consumer goods manufacturers and government regulatory agencies have long sought ways to provide ingredient information to a consumer in an accurate yet informative way. However, limited space on product packaging and labels inhibits the amount of information that can be provided to the consumer. It has now been surprisingly discovered that providing additional ingredient information on an ingredient label can be done in a way that enables more efficient use of limited label space and improves product sales. In particular, it has been discovered that configuring the label to have certain dimensions and/or positioning the ingredient information on the label in a specific way allows ingredient information to be presented to a consumer in a user-friendly way, while still complying with the strict government labeling requirements for cosmetic products. Surprisingly, it has been found that a consumer may be more likely to buy a product with this type of ingredient label configuration, especially when the label provides additional information for active ingredients and/or ingredients that are present in appreciable amounts (e.g., greater than 0.5%).

As used in the description and the appended claims, the singular forms “a”, “an”, and “the” are intended to include the plural forms as well, unless the context clearly indicates otherwise. All ingredient percentages disclosed herein are by weight of the total composition, unless specifically stated otherwise. All ratios are weight ratios, unless specifically stated otherwise. The number of significant digits conveys neither a limitation on the indicated amounts nor on the accuracy of the measurements. All numerical amounts are understood to be modified by the word “about” unless otherwise specifically indicated. All measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified. All numeric ranges are combinable and include each value in the delineated range. Delineated upper and lower range limits are interchangeable to create further ranges not explicitly delineated.

The embodiments disclosed herein can comprise, consist essentially of, or consist of, the essential components as well as optional elements described herein. As used herein, “consisting essentially of” means that the embodiment or component may include additional ingredients, but only if the additional ingredients do not materially alter the basic and novel characteristics of the claimed embodiment.

### Definitions

“About” modifies a particular value by referring to a range equal to plus or minus twenty percent (+/−20%) or less (e.g., less than 15%, 10%, or even less than 5%) of the stated value.

“Active ingredient” means a compound(s) that, when applied to target surface (e.g., keratinous tissue), provides a benefit or improvement.

“Additional ingredient information” means any information other than a formal ingredient name or an informal ingredient name that is provided on a product package in a retail environment and related to an ingredient in the product.

“Cosmetic” means a product intended to be applied to the human body for cleansing, beautifying, promoting attractiveness, or altering the appearance of a person, which does not require a prescription or administration by a licensed medical professional.

“Dermatologically acceptable” means that the compositions or components thereof so described are suitable for use in contact with mammalian keratinous tissue without undue toxicity, incompatibility, instability, allergic response, and the like.

“Disposed” means an element is positioned in a particular place relative to another element.

“Effective amount” means an amount of a compound or composition sufficient to significantly induce a positive benefit to keratinous tissue, such as a health, appearance, and/or feel benefit, including, independently or in combination, the benefits disclosed herein, but low enough to avoid serious side effects (i.e., to provide a reasonable benefit to risk ratio, within the scope of sound judgment of the skilled artisan). An effective amount of an active ingredient is an amount of sufficient to regulate a desired condition of mammalian keratinous tissue when topically applied thereto in a personal care composition over the course of a treatment period.

“Formal ingredient name” refers to an ingredient name from a uniform system of labeling names that are nationally and/or internationally recognized for use in the subject product. For example, in cosmetic products, the formal ingredient name is the International Nomenclature Cosmetic Ingredient (“INCI”) name of the cosmetic ingredient as set forth by the International Nomenclature Committee and published by the Personal Care Products Council in the International Cosmetic Ingredient Dictionary and Handbook.

“Informal ingredient name” refers to the name of an ingredient other than its formal ingredient name.

“Ingredient label” means any written or printed information distributed with a product that lists at least some of the ingredients in the product. For example, an ingredient label may be in the form of a list of product ingredients printed directly on the primary and/or secondary packaging of the product.

“Personal care composition” means a topical composition for regulating a condition of mammalian keratinous tissue (e.g., skin, hair, fingernails). Some nonlimiting examples of personal care compositions include skin creams, lotions, serums, shave prep compositions, body washes, deodorants and antiperspirants, shampoos, conditioners, toothpaste, tooth whiteners, oral rinses, combinations of these and the like.

“Primary packaging” means packaging in direct contact with the product contained therein. A nonlimiting example of a primary package is a jar that contains a facial skin moisturizer.

“Secondary packaging” means packaging that contains the primary package. A nonlimiting example of a secondary package is a box that contains a jar of facial skin moisturizer.

“Skin care” refers to regulating and/or improving a skin condition. Some nonlimiting examples of skin care products include skin creams, skin serums, skin moisturizers, skin lotions, facial cleansers, and body washes.

“Topical” refers to a composition that is intended to be applied to a bodily surface such as skin or hair.

### Ingredient Label

Most, if not all, personal care products are required by government regulations to have an ingredient label that is visible to consumers. In some instances, the regulations governing ingredient labels can be strict, especially with regard to ingredient labels for cosmetic products that are topically applied to keratinous tissue such as skin and hair. The ingredient labels described herein function with the ingredient list on the label to provide a user-friendly, informative ingredient list to a consumer that complies with applicable labeling requirements. In other words, the list(s) of ingredients exploit the configuration of the label to provide additional ingredient information to a consumer compared to a conventional ingredient label.

The personal care products herein include a personal care composition disposed in a primary package such as a jar, bottle, or other suitable package known for containing personal care compositions of the type and an ingredient label disposed on the primary package. In some instances, the primary package containing the personal care composition may be disposed in a secondary package such as a box. The ingredient label may be located on the primary package and/or secondary package. The ingredient label may be printed directly on the product package (primary and/or secondary) or printed on a suitable substrate that is affixed to the package, e.g., using a suitable adhesive. The material used to make the package and/or label substrate is not particularly limited and may be any suitable material known in the art, as long as the information on the ingredient label is discernible by a person with 20/20 vision at a distance of between 12 and 18 inches under normal daytime lighting conditions. It is to be appreciated that the present disclosure also contemplates an ingredient label in the form of an insert, riser, display pack, leaflet, promotional literature or any other written or printed information distributed with the personal care product.

The present ingredient label and the ingredient information contained thereon are sized and positioned on the package to comply with all applicable government regulations and to present additional ingredient information desired by consumers. For cosmetic compositions, the ingredient label should be displayed prominently under customary conditions of purchase. For example, the ingredient label of a cosmetic skin care or hair care product should be displayed on the top or side of the primary and/or secondary package and not the bottom.

The package displaying the ingredient label to a consumer may have a total surface area of less than 650 cm² (e.g., less than 520 cm², 390 cm², 320 cm², 260 cm², or even less than 200 cm²), but typically greater than 65 cm². For example, the label may be located on a secondary package in the form of a box comprising 6 sides (i.e., 2 pairs of opposing side panels, a top panel and a bottom panel), each side having a surface area of between 15 cm² and 160 cm² (e.g., between 20 cm² and 125 cm², 25 cm² and 100 cm², or even between 30 cm² and 75 cm²). In another example, the label may be located on the primary package in the form of a small jar that has one continuous side, and the portion of the jar containing the ingredient list may be between 10% and 75% (e.g., between 15% and 70%, 20% and 60%, or even between 25% and 50%) of the total surface area of the jar. Of course, it is to be appreciated that the primary and/or secondary package may be provided in any shape, with one or more equal or non-equal sides, as desired.

The ingredient labels described herein are particularly useful for products that have 10 or more ingredients (e.g., between 10 and 50 ingredients, between 12 and 40 ingredients, or even between 15 and 30 ingredients) due to the space limitations encountered on the label and/or package. To comply with government regulations and industry standards, it may be desirable for the letters and numbers used in the written information on the ingredient label to be 0.7 mm or more in height (e.g., at least 0.75 mm, 0.79 mm, 1.50 mm, 2.00 mm, 2.50 mm, 3.00 mm, 4.00 mm, 5.00 mm, or even at least 7.00 mm), but generally less than 1 cm in height. If the labeling surface is less than 77.4 cm², then it may be desirable to ensure that the letters on the ingredient label are at least 0.79 mm in height. If the labeling surface is 77.4 cm² or more, then it may be desirable to ensure that the letters on the ingredient label are at least 1.5 mm in height.

The ingredient label herein includes a first portion and a second portion for displaying ingredient information. The first portion of the ingredient label includes a list of formal ingredient names that correspond to some or all of the ingredients in the composition. In some instances, the list of formal ingredient names includes only those ingredients that are present in the composition at an amount of at least 0.1% (e.g., ingredients present at 0.5% or more, 1% or more, 2% or more, 3% or more, 4% or more, or 5% or more). The second portion of the ingredient label includes a list of informal ingredient names and/or additional ingredient information such as an ingredient function, ingredient benefit, and/or sourcing information (e.g., whether the ingredient is naturally, sustainably, and/or responsibly sourced). The informal ingredient name and/or additional ingredient information is positioned proximate the corresponding formal ingredient name such that a consumer can recognize the association between the formal ingredient name and the informal ingredient name and/or additional ingredient information.

In some instances, it may be desirable to highlight certain ingredient information in the first and/or second portions of the ingredient label to identify ingredients that are naturally, sustainably, and/or responsibly sourced. The ingredients may be highlighted by any suitable means known in the art (e.g., asterisk, caret, coloring, shading, bolding, italics, and combinations of these). In some instances, the means of highlighting may be selected to communicate additional information to a consumer. For example, the ingredient information may be highlighted using a symbol or color that consumers typically associate with naturally, sustainably, and/or responsibly sourced products (e.g., a tree, the Earth, a panda, a dolphin, the color green). Additionally or alternatively, the ingredient label may include a third portion where additional ingredient information corresponding to the highlighted ingredient information is provided. In some instances, the additional ingredient information related to the highlighted ingredient may be displayed on another portion of the package.

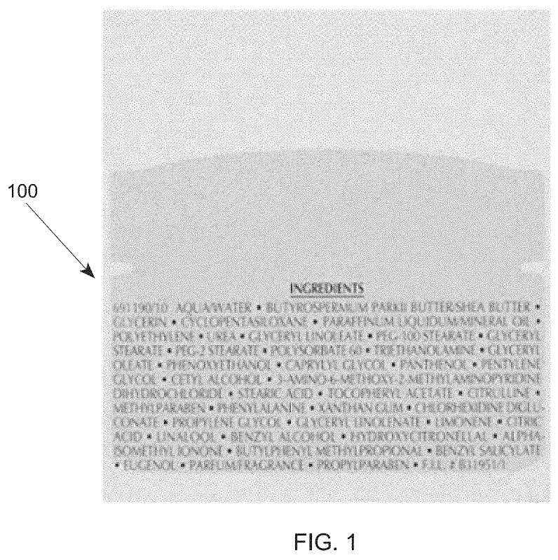

FIG. 1 shows a conventional ingredient label 100 for a cosmetic skin care product. As can be seen in FIG. 1, only the formal ingredient name of each ingredient in the product is displayed on the ingredient label.

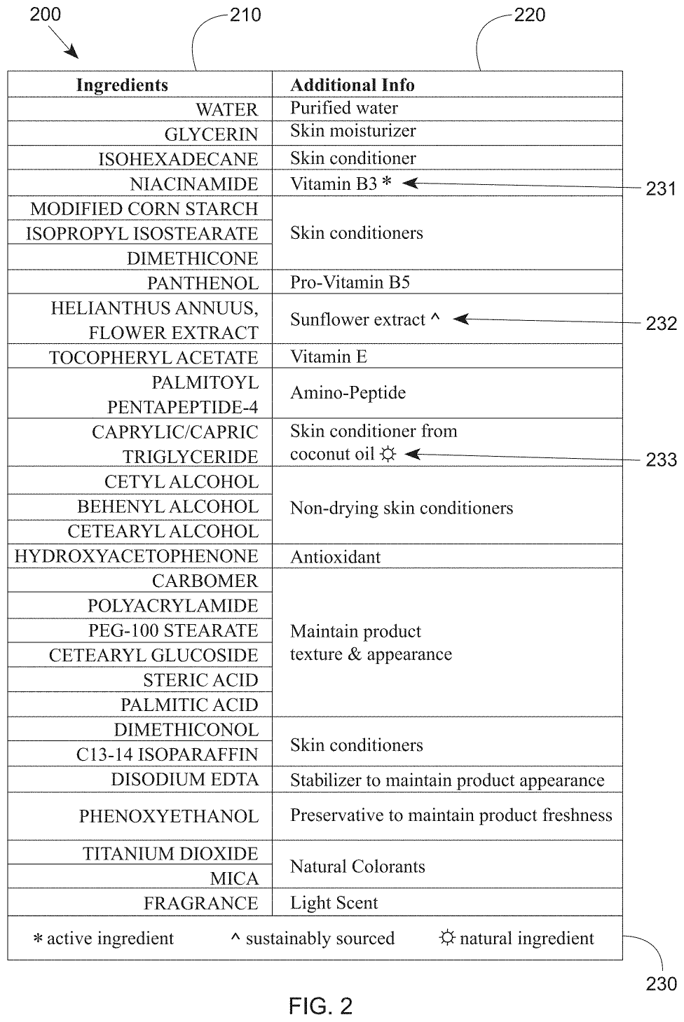

In contrast to the conventional label arrangement of FIG. 1, FIG. 2 illustrates an example of an embodiment of the present ingredient label 200 for a cosmetic skin care product. The ingredient label 200 shown in FIG. 2 includes a first portion 210 and a second portion 220. The first portion 210 and second portion 220 are arranged as columns in a side-by-side configuration. As can be seen in FIG. 2, the ingredient information displayed in the columns of the first portion 210 and the second portion 220 may be arranged in rows such that a consumer can more easily associate a formal ingredient name to its corresponding informal ingredient name and/or additional ingredient information. In some instances, it may be desirable to associate multiple rows in the first portion 210 to a single row in the second portion, or vice versa, which may use the available label space more effectively. Such a configuration may be particularly desirable when the product contains multiple ingredients that perform the same function and/or if one or more of the formal ingredient name, informal ingredient name, and ingredient function are long (e.g., include more than 15 letters).

As illustrated in FIG. 2, the first portion 210 of the ingredient label 200 includes the formal ingredient names for each ingredient in the product and the second portion 220 includes an informal ingredient name and/or additional ingredient information adjacent the formal ingredient name and in the same row(s). For example, Helianthus annuus Flower Extract is a formal ingredient name (i.e., an INCI name) and “sunflower extract” is an informal ingredient name. In another example, Glycerin is a formal ingredient name and “skin moisturizer” is a function. In still another example, Titanium Dioxide is a formal ingredient name and “natural colorants” can be an informal ingredient name, a function, and/or sourcing information. The second portion 220 of the ingredient label 200 also includes highlighted ingredient information. As shown in FIG. 2, the highlighted ingredient information is highlighted with an asterisk 231 or a caret 232. Of course, it is to be appreciated that other highlighting the ingredient information may be used. For example, the naturally, sustainably, and/or responsibly sourced ingredients in the product may be shown in green lettering or the image of a sun 233. In some instances, the ingredient label 200 may include an optional third portion 230 where additional ingredient information (e.g., naturally, sustainably, or responsibly sourced) related to the highlighted ingredient can be displayed.

In some instances, labeling regulations and/or industry standards may require listing the ingredients in the product from highest concentration to lowest concentration. In these instances, it may be desirable and/or necessary to an informal ingredient name in two or more separate locations on the ingredient label 200. For example, isohexadecane and dimethiconol are both skin conditioners, but are spatially separated from one another on the ingredient label 200 illustrated in FIG. 2.

The first portion 210 and second portion 210 of the ingredient label 200 may be the same or different sizes. For example, the first portion 210 and second portion 220 may have a ratio of first portion surface area to second portion surface area of 1:1, as illustrated in FIG. 2. However, in some instances, it may be desirable to configure the first portion 210 to be smaller than the second portion 220 so that more information can be displayed in the second portion 220 of the ingredient label 200. This can be important for ingredients that have complex functions, more than one common name, or when more than one kind of ingredient information is provided (e.g., common ingredient name and one or more ingredient functions and/or sources). Thus, the ratio of first portion surface area to second portion surface area may range from 2:3 to 1:5.

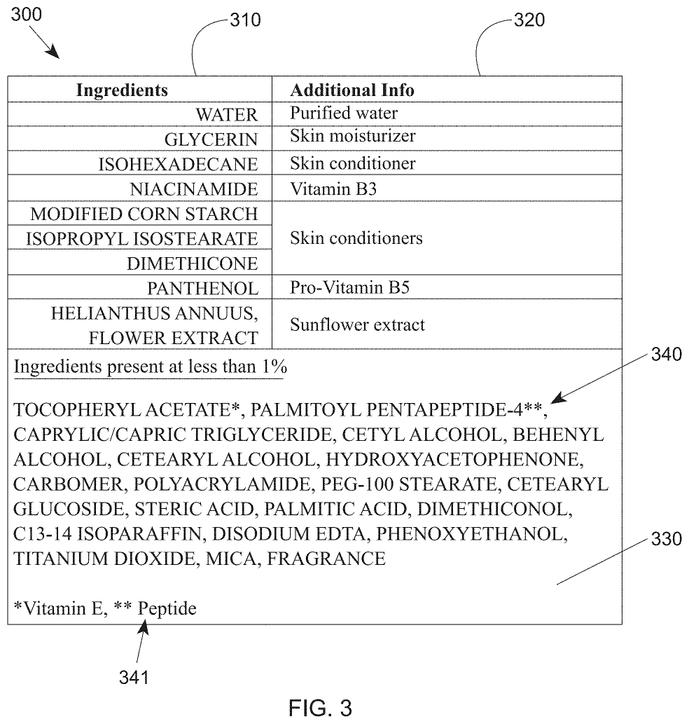

FIG. 3 shows another example of an embodiment of the present ingredient label 300. As can be seen in FIG. 3, the ingredient label 300 includes a first portion 310, a second portion 320 and a third portion 330. The first portion 310 and second portion 320 are arranged as columns in a side-by-side configuration with the third portion 330 disposed underneath the first and second portions 310 and 320. The ingredient information displayed in the first portion 310 and second portion 320 columns may be arranged in rows as described above for FIG. 2. In this example, the first and second portions 310 and 320 of the ingredient label 300 only display ingredient information for ingredients present in the product at an amount of 1% or more. As can be seen in FIG. 3, the third portion 330 of the ingredient label includes formal ingredient names for the ingredients in the product that are present at less than 1%. In some instances, it may be desirable to highlight one or more formal ingredient names in the third portion 330 and provide a related informal ingredient name and/or additional ingredient information in another portion of the label and/or third portion 330. For example, as can be seen in FIG. 3, the formal ingredient name Palmitoyl Pentapeptide-4 is highlighted with two asterisks 340. The informal ingredient name “Peptide” 341 is then displayed below the list of formal ingredient names in the third portion 330 of the ingredient label 300. The informal ingredient name 341 is also highlighted with two asterisks to enable a consumer to associate the highlighted formal ingredient name with its corresponding informal ingredient name and/or ingredient additional ingredient information.

The first portion 310, second portion 320, and third portion 330 of the ingredient label 300 may all be the same size or different sizes. In some instances, it may be desirable to configure the first portion 310 of the ingredient label 300 to be smaller than the second portion 320 and/or the third portion 330 so that more information can be displayed in the second and/or third portions 320 and/or 330 of the ingredient label 300.

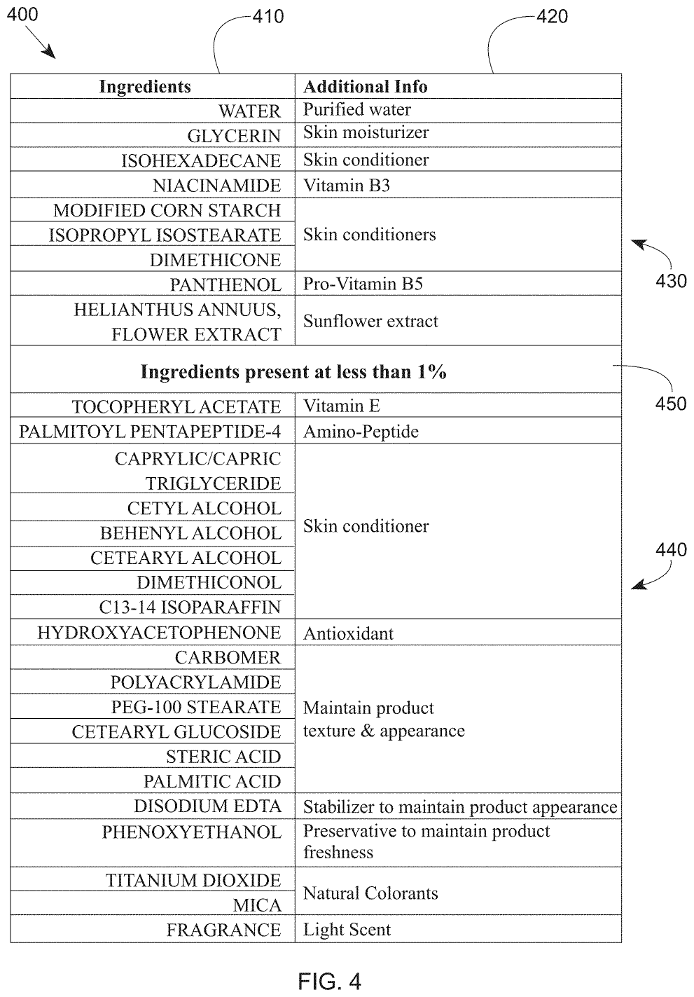

FIG. 4 illustrates another example of the present ingredient label 400. The ingredient label 400 shown in FIG. 4 is arranged in two side-by-side columns 410 and 420. The first column 410 includes formal ingredient names and the second column 420 includes informal ingredient names and/or additional ingredient information. The ingredient label 400 also includes an upper portion 430 a lower portion 440. The upper portion 430 contains ingredient information (i.e., formal ingredient names, informal ingredient names, and additional ingredient information) for ingredients present in the product at a concentration of 1% or greater, by weight. The lower portion 440 contains ingredient information for ingredients present in the product at concentrations of less than 1%. In some instances, the upper and lower portions 430 and 440 of the ingredient label 400 may be separated by a third portion 450 that indicates the concentrations of the ingredients in the upper and lower portions 430 and 440.

Cosmetic product ingredients are generally ordered from highest concentration to lowest concentration on the ingredient label, usually due to government regulations or an industry standard. However, in some instances, the regulation or standard may only apply to ingredients that are present at or above a certain threshold amount (e.g., 0.1%, 0.5%, or 1%). By configuring the ingredient label with an upper and lower portion 430 and 440, as shown in FIG. 4, the ingredients in the lower portion 430 may be ordered differently from the ingredients in the upper portion 440. For example, the ingredients in the lower portion 440 may be ordered according to a common feature such that ingredients that share the common feature are listed together in the first column 410 and the shared feature (e.g., informal ingredient name, function, or sourcing) need only be provided once in the second column 420 for all the ingredients. Ordering the ingredient information in this way may enable more efficient use of the available space on a small ingredient label while improving user-friendliness.

While the present ingredient label is exemplified for use with personal care compositions such as cosmetic skin care product, hair care, grooming products, and oral care products (e.g., toothpaste, tooth whiteners, and oral rinses), it is to be appreciated that the present ingredient label can also be used to great advantage with other products where consumers desire more information about the ingredients in the product (e.g., personal health compositions such as vitamin and mineral supplements, laundry products, dishwashing products, hard surface cleaners, food, drinks, and medicine).

### EXAMPLES

### Example 1

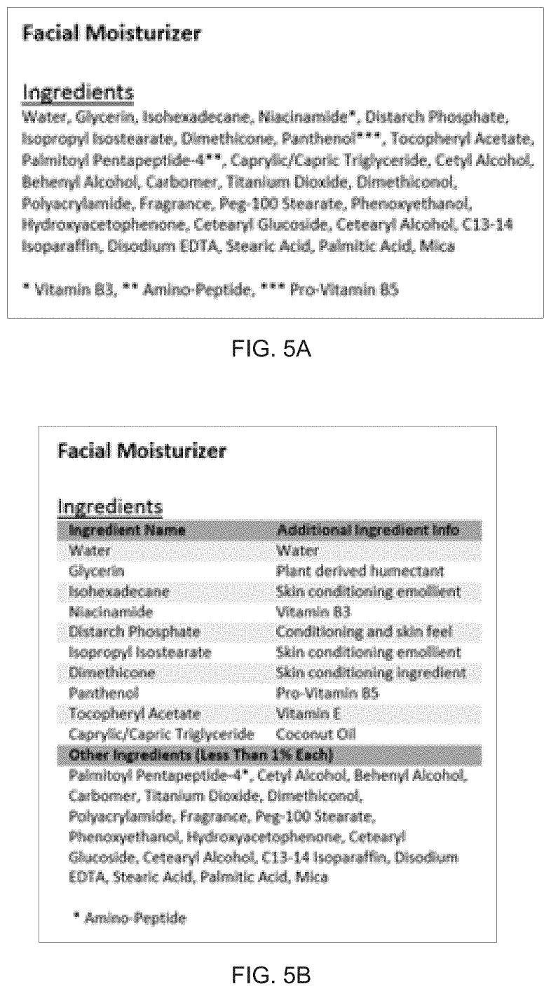

This example demonstrates the consumer preference for products with the present ingredient label versus products with a conventional ingredient label. A study was conducted in which 2000 panelists were shown the conventional ingredient label illustrated in FIG. 5A and the inventive ingredient label illustrated in FIG. 5B simultaneously. As can be seen in the figures, both ingredient labels list the same formal ingredient names. However, the ingredient label in FIG. 5A highlights 3 active ingredients whereas the ingredient label in FIG. 5B only highlights 1 active ingredient. Also, the ingredient label in FIG. 5A may appear to include fewer ingredients than the ingredient label in FIG. 5B due to the additional ingredient information provided in the label of FIG. 5B. The test panelists were then asked which product they would buy.

Surprisingly, twenty-six percent of the test panelists indicated they would buy the product with the ingredient label in FIG. 5A, while seventy-four percent indicated they would buy the product with the ingredient label in FIG. 5B. These results are surprising because the test panelists were expected to choose a product with an ingredient label that highlights the most active ingredients and/or appears to have fewer ingredients in the product.

### Example 2

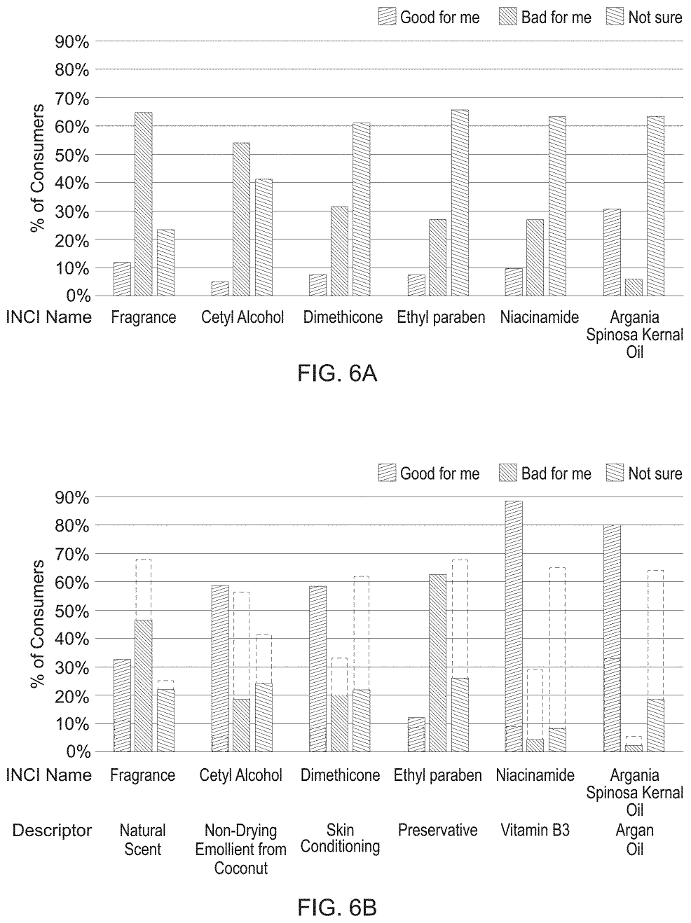

This example demonstrates how providing additional ingredient information on an ingredient label can influence consumer perception of a product. In this study, 80 panelists were presented with a list of ingredients commonly found in cosmetic skin care compositions. The panelists were first presented with just the list of ingredients and asked to identify each ingredient in the list as “good for me”, “bad for me”, or “not sure”. The chart in FIG. 6A illustrates the test panelists' perception of 6 ingredients from the list of ingredients provided to the panelists. The panelists were then given a second list of the same ingredients, except that the second list of ingredients included a descriptor for each ingredient. The descriptor provided additional ingredient information in the form of an informal ingredient name or ingredient function. The chart in FIG. 6B illustrates the panelists' perception of the 6 ingredients after seeing the descriptor. As illustrated in FIGS. 6A and 6B, when the test panelists were presented with the additional ingredient information, there was a dramatic shift from “not sure” to “good for me” for niacinamide and argan oil and a shift from “not sure” to “bad for me” for ethyl paraben. Niacinamide presented a particularly striking example because it is a well-known skin care active, and yet only 8% of panelists thought it was good for them after being presented with the first list of ingredients. However, after seeing the product descriptor, “Vitamin E”, in the second list of ingredients, 88% of the test panelists identified niacinamide as “good for me”. For fragrance, cetyl alcohol, and dimethicone there was a significant shift in test panelist perception from “bad for me” to “good for me,” when presented with the additional ingredient information. Thus, the results of this study suggest that consumer perception of a product and intent to purchase the product can be influenced by providing an ingredient label that includes consumer-relevant, user-friendly ingredient information.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care product, comprising:
a) a personal care composition disposed in a package; and
b) an ingredient label disposed on the package, wherein the ingredient label includes:
i) a first portion containing a formal ingredient name for each ingredient in the product, and
ii) a second portion containing at least one of an informal ingredient name and additional ingredient information that relates to the formal ingredient names in the first portion.

2. The personal care product of claim 1, wherein the personal care product is a cosmetic skin care product.

3. The personal care product of claim 1, wherein the personal care composition contains 10 or more ingredients.

4. The personal care product of claim 1, wherein the ingredient label has a surface area of less than about 65 cm².

5. The personal care product of claim 1, wherein the first portion of the ingredient label occupies about 20% to 50% of the surface area of the ingredient label.

6. The personal care product of claim 5, wherein a ratio of the surface area of the first portion to the second portion is between 1:1 and 1:5.

7. The personal care product of claim 1, wherein the first and second portions of the ingredient label are arranged as discrete columns in a side-by-side configuration, and the formal ingredient name and its related informal ingredient name and/or additional ingredient information are positioned adjacent one another in their respective columns.

8. The personal care product of claim 1, wherein the ingredient label further comprises an upper portion and a lower portion, and ingredient information displayed in the upper portion relates only to ingredients present in the personal care composition at a concentration of 1% or more, and wherein the ingredient information is ordered according to ingredient concentration.

9. The personal care product of claim 8, wherein ingredient information displayed in the lower portion relates only to ingredients present in the personal care composition at less than 1% by weight of the composition, and at least some of the ingredient information is ordered according to a common characteristic shared by two or more ingredients.

10. The personal care product of claim 9, wherein the common characteristic is selected from the group consisting of an ingredient function, an informal ingredient name, and an ingredient source.

11. The personal care product of claim 1, wherein the product is in a brick-and-mortar retail environment.

12. A personal care product, comprising:
a) a personal care composition disposed in a package; and
b) an ingredient label disposed on the package, wherein the ingredient label includes:
i) a first portion containing a list of formal ingredient names, wherein each formal ingredient name in the first portion list designates an ingredient present in the personal care composition at an amount of 0.5% or more,
ii) a second portion containing at least one of an informal ingredient name and additional ingredient information that relates to each formal ingredient name in the first portion, and
iii) a third portion containing a list of formal ingredient names, wherein each formal ingredient name in the third portion designates an ingredient present in the personal care composition at an amount of less than 0.5%.

13. The personal care product of claim 12, wherein the personal care composition contains 10 or more ingredients.

14. The personal care product of claim 12, wherein the ingredient label has a surface area of less than about 65 cm².

15. The personal care product of claim 12, wherein the first portion and the second portion of the ingredient label occupy about 30% to 95% of the surface area of the ingredient label.

16. The personal care product of claim 12, wherein formal ingredient names of active ingredients in the third portion of the ingredient label are highlighted, and at least one of an informal ingredient name and additional ingredient information of the active ingredient is provided below the list of formal ingredient names in the third portion.

17. The personal care product of claim 12, wherein the first portion and the second portions are arranged as discrete columns in a side-by-side configuration, and the formal ingredient name and its related informal ingredient name and/or additional ingredient information are position adjacent one another in their respective columns.
